(19) United States
(12) Patent Application Publication
(10) Pub. No.: US 2016/0324999 A1
(43) Pub. Date: Nov. 10, 2016

(54) PERFUMED PAPER ROLL AND MANUFACTURING METHOD THEREOF

(71) Applicant: Ching-Lien KUAN SHEN, TAIPEI CITY (TW)

(72) Inventor: Ching-Lien KUAN SHEN, TAIPEI CITY (TW)

(21) Appl. No.: 15/145,811

(22) Filed: May 4, 2016

(30) Foreign Application Priority Data
May 6, 2015 (TW) 104114471

(51) Int. Cl. A61L 9/012

(52) U.S. Cl. CPC A61L 9/012

(57) ABSTRACT

A perfumed paper roll has a plurality of rings of paper body. Adhesive is between rings of the paper body. The rings of the paper body are impregnated with liquid perfume. A method of manufacturing a perfumed paper roll includes: winding a paper strip by utilizing a rotating machine to form a paper roll having a plurality of rings of paper body; dispensing adhesive between adjacent rings of the paper body during a rotating process; and dripping liquid perfume on the paper roll after removing the paper roll to form the perfumed paper roll.

### RELATED APPLICATIONS

This application claims priority to Taiwan Application Serial Number 104114471 filed May 6, 2015, which is herein incorporated by reference.

### BACKGROUND

1. Field of Invention

The present invention relates to a paper roll. More particularly, the present invention relates to a perfumed paper roll and a manufacturing method thereof.

2. Description of Related Art

Perfume has the effects of improving environmental sanitation, removing odor, purifying air, etc.

Common perfume exists in a liquid form. However, such liquid perfume is easily spilt and turned over during transportation and when being used, which is very inconvenient.

For the forgoing reason, there is a need to solve the above-mentioned problem.

### SUMMARY

The present invention provides a perfumed paper roll to avoid liquid perfume being spilt and turned over when being used.

The present invention also provides a manufacturing method of a perfumed paper roll so as to manufacture a perfumed paper roll by utilizing a simple method.

According to one aspect of the present invention, a perfumed paper roll is provided. The perfumed paper roll comprises a plurality of rings of paper body, and each ring of the paper body is impregnated with liquid perfume.

According to one embodiment of the present invention, adhesive is between the rings of the paper body.

According to another aspect of the present invention, the invention provides a method of manufacturing a perfumed paper roll. The method comprises the steps of: fixing a rod on a rotating part of a rotating machine; preparing a paper strip having a required length and a required width, and winding one end of the paper strip around the rod for one turn to form a first ring of paper body; continuously winding the paper strip to form a second ring of the paper body, and dispensing adhesive between the second ring of the paper body and the first ring of the paper body; activating the rotating machine, winding the paper strip around the rod to form a paper roll having a plurality of rings of the paper body, and dispensing the adhesive between adjacent rings of the paper body of the paper roll during a rotating process; and removing the paper roll from the rod, and dripping liquid perfume on the paper roll to form the perfumed paper roll.

In the perfumed paper roll according to one embodiment of the present invention, each ring of the paper body of the paper roll is impregnated with liquid perfume. Hence, there is no concern that the liquid perfume is spilt and turned over and the whole perfumed paper roll is convenient to carry and transport.

It is to be understood that both the foregoing general description and the following detailed description are by examples, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 depicts an outside view of a perfumed paper roll according to one embodiment of this invention; and

FIG. 2 depicts a flowchart of manufacturing a perfumed paper roll according to one embodiment or this invention.

### DESCRIPTION OF THE EMBODIMENTS

Reference will now be made in detail to the present embodiments of the invention, examples of which are illustrated in the accompanying drawings. Wherever possible, the same reference numbers are used in the drawings and the description to refer to the same or like parts.

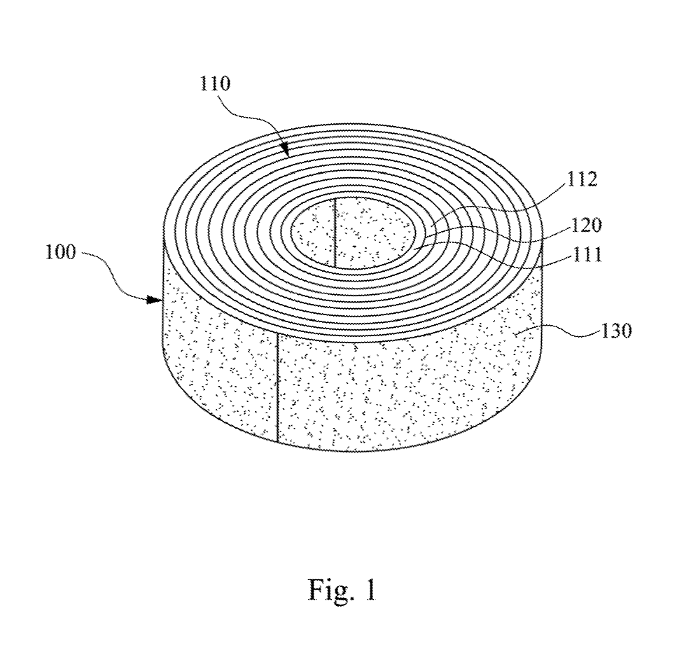

A description is provided with reference to FIG. 1. According to one embodiment of the present invention, a perfumed paper roll 100 has a plurality of rings of paper body 110. Adhesive 120 is between the rings of the paper body so as to bond adjacent rings of the paper body. Thus, the whole paper roll will hold its shape or will not deform. In addition, the each ring of the paper body is impregnated with liquid perfume 130. When the perfumed paper roll 100 is used, the liquid perfume 130 in the perfumed paper roll 100 is blown by a fan of a diffuser or natural air flow to vaporize and dissipate.

In a perfumed paper roll according to one embodiment of the present invention, each ring of paper body of a paper roll is impregnated with liquid perfume. That is, the paper roll serves as a carrier of the liquid perfume. The paper roll has excellent capillary absorption capacity so there is no concern that the liquid perfume is spilt and turned over and the whole perfumed paper roll is convenient to carry and transport.

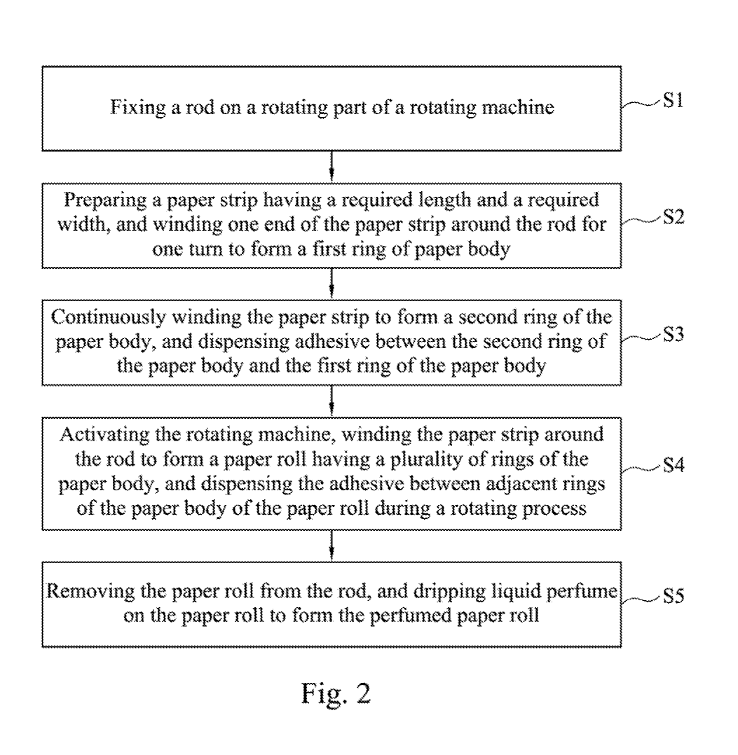

A description is provided with reference to FIG. 1 and FIG. 2. FIG. 2 depicts a flowchart of manufacturing a perfumed paper roll according to one embodiment or this invention, which is used for illustrating manufacturing steps for the perfumed paper roll. The manufacturing steps comprise: (1) fixing a rod (not shown) on a rotating part of a rotating machine (not shown) (S1); (2) preparing a paper strip having a required length and a required width, winding one end of the paper strip around the rod for one turn to form a first ring of paper body 111 (S2); continuously winding the paper strip to form a second ring of t he paper body 112, and dispensing the adhesive 120 between the second ring of the paper body 112 and the first ring of the paper body 111 (S3); activating the rotating machine, winding the paper strip around the rod to form a paper roll having the plurality of rings of the paper body 110, and dispensing the adhesive 120 between adjacent rings of the paper body of the paper roll during a rotating process (S4); removing the paper roll from the rod, and dripping the liquid perfume 130 on the paper roll to form the perfumed paper roll 100 (S5).

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A perfumed paper roll comprising a plurality of rings of paper body, wherein the rings of the paper body are impregnated with liquid perfume.

2. The perfumed paper roll of claim 1, wherein adhesive is dispensed between the rings of the paper body.

3. A method of manufacturing a perfumed paper roll, comprising the steps of:
fixing a rod on a rotating part of a rotating machine;
preparing a paper strip having a required length and a required
width, and winding one end of the paper strip around the rod for one turn to form a first ring of paper body;
continuously winding the paper strip to form a second ring of the paper body, wherein a adhesive is dispensed between the second ring of the paper body and the first ring of the paper body;
activating the rotating machine, winding the paper strip around the rod to form a paper roll having a plurality of rings of the paper body, and dispensing the adhesive between adjacent rings of the paper body of the paper roll during a rotating process; and
removing the paper roll from the rod, and dripping liquid perfume on the paper roll to form the perfumed paper roll.
